(19) United States
(12) Patent Application Publication
(10) Pub. No.: US 2014/0080824 A1
(43) Pub. Date: Mar. 20, 2014

(54) STRUCTURE-GUIDED IDENTIFICATION OF BINDING INTERACTIONS OF HUMAN LAMININ RECEPTOR PRECURSOR WITH LAMININ AND IDENTIFICATION OF COMPOUNDS THAT AFFECT BINDING

(71) Applicants: Daniel Meruelo, Scarborough, NY (US); Kelly Jamieson, New York, NY (US); Vincent DiGiacomo, New York, NY (US)

(72) Inventors: Daniel Meruelo, Scarborough, NY (US); Kelly Jamieson, New York, NY (US); Vincent DiGiacomo, New York, NY (US)

(73) Assignee: NEW YORK UNIVERSITY, New York, NY (US)

(21) Appl. No.: 13/880,872

(22) PCT Filed: Oct. 20, 2011
(86) PCT No.: PCT/US11/57120

§ 371 Date: Dec. 3, 2013

Related U.S. Application Data
(60) Provisional application No. 61/405,578, filed on Oct. 21, 2010.

(51) Int. Cl. C07D 295/194; C07D 241/46

(52) U.S. Cl. CPC C07D 295/194; C07D 241/46
USPC 514/232.8; 514/250

(57) ABSTRACT

The present invention pertains to compounds which interfere with the binding of laminin to the laminin receptor (LamR). Such compounds are useful for treating diseases such as cancer, Alzheimer's Disease, and certain viral and bacterial infections.

This application claims priority under 35 USC Section 119(e) from Provisional application No. 61/405,578 filed Oct. 21, 2010. The entire disclosure of said application is incorporated by reference in its entirety.

The United States Government has certain rights to this invention by virtue of funding received from U.S. Public Health Service grant numbers CA 100687 and CA 68498 from the National Cancer Institute, National Institutes of Health and Department of Health and Human Services

### FIELD OF THE INVENTION

The present invention pertains to compounds which interfere with the binding of laminin to the laminin receptor (LamR). Such compounds are useful for treating diseases such as cancer, Alzheimer's Disease, and certain viral and bacterial infections.

### BACKGROUND OF THE INVENTION

The 37/67 kDa human laminin receptor (LamR) is a cell surface receptor for laminin, prion protein, and a variety of viruses. Because of its wide range of ligands, LamR plays a role in numerous pathologies. LamR overexpression in cancer correlates with a highly invasive cell phenotype and increased metastatic ability, mediated by interactions between LamR and laminin. The specific targeting of LamR with small-interfering RNA (siRNA), blocking antibodies, competitive peptides and Sindbis viral vectors have been associated with antitumor effects.

LamR was originally identified as a non-integrin cell surface receptor for the extracellular matrix molecule laminin (1-3). Laminins, other glycoproteins, collagen IV and proteoglycans constitute a tight network to form the basement membrane. Laminin-1, a 900 kDa glycoprotein, contains many bioactive domains involved in binding both integrin and non-integrin receptors (4) and is vital for basement membrane assembly (5). Interactions between LamR and laminin play a major role in mediating changes in the cellular environment that affect cell adhesion (6), neurite outgrowth (4), and tumor growth and metastasis (7).

Overexpression of LamR has been shown in many cancers, including lung (8), breast (9), gastric (10), colon (11, 12), ovarian (13, 14), uterine (15), thyroid (16), prostate (17), liver (18) and melanoma (19, 20). This over-expression is associated with an invasive phenotype and metastatic ability (21-25). Interactions between LamR and laminin contribute to tumor cell attachment to the basement membrane. These properties render LamR a prognostic factor in determining the degree of malignancy in human cancer patients (21, 26, 27). Understanding how LamR and laminin interact provides insight into tumor invasion and metastasis.

In addition to its laminin binding function, LamR has been observed to act as the cell surface receptor for pathogenic prion protein and a variety of viruses, including Sindbis virus (28-30). LamR has also been reported to bind EGCG, a polyphenol found in green tea (31). Interestingly, LamR also has a dual function as a component of the translation machinery. Both human LamR and the p40 ribosomal protein are encoded by the same gene, 37LRP/p40 (32). Intracellular LamR is found on the 40S ribosome and polysomes (33, 34) and in the nucleus (35), which suggests that LamR is a multifunctional protein. As a testament to its diverse functions, silencing LamR expression in mammalian cells in vitro induces G1 cell cycle arrest, inhibition of migration, loss of viability, shutdown of protein translation, and loss of LamR association with the polysomes (34).

Sequence conservation of 37LRP/p40 genes across species has been demonstrated, with evolution of the C-terminal tail convergent with vertebrates (32, 36-42). At the cell surface, LamR, which is targeted to the membrane via fatty-acid acylation (43), exists as both a monomer (37 kDa) and a dimer (67 kDa)(44). Although the homo- or hetero-dimeric state of LamR has not been fully resolved, both 37 kDa and 67 kDa LamR bind laminin (45, 46).

Characterization of LamR interactions with laminin has not been fully conclusive. Several LamR segments, including residues 161-180 (peptide G)(47), residues 205-229(48), and TEDWS-containing C [symbol] terminal repeats (49) have been implicated as binding epitopes for laminin. A high-resolution crystal structure of the majority of LamR, residues 1 through 220 (abbreviated LamR220) (46) (PDB code 3BCH) has been reported (U.S. patent application Ser. No. 12/181,653). It has been previously determined that LamR220 binds laminin with similar affinity as full-length LamR and inhibits Sindbis virus infection in vitro (46). Full knowledge about the LamR/laminin interaction with the ability to specifically disrupt the interaction has therapeutic value.

U.S. Pat. No. 7,306,792 discloses methods for treating tumors which express greater amounts of High Affinity Laminin Receptors (HALR) on their cell surface than normal cells of the same lineage comprising systemically administering effective antitumor amounts of defective Sindbis Virus Vectors.

Co-pending application Ser. No. 12/390,096 discloses treating tumors which express greater amounts of HALR on their cell surface than normal cells of the same lineage comprising administering effective antitumor amounts of Replication Competent Sindbis Virus Vectors.

U.S. Pat. No. 7,303,998 discloses defective Sindbis viral vectors and methods for treating tumors which express greater amounts of HALR than normal cells of the same lineage comprising systemically administering the vectors.

Co-pending application Ser. No. 12/181,653 discloses a human laminin receptor Crystal structure and methods for using various models and means for the development of novel therapeutics that block and/or mimic laminin receptor interactions in the context of Alzheimer's Disease, diagnosis of other neurological disorders, cancer and viral and bacterial infections.

What is needed in the art are compounds and methods of use thereof for treating individuals suffering from diseases which are mediated by the laminin receptor (LamR) such as cancer, Alzheimer's Disease, certain bacterial and viral infections and diseases involving prions.

### SUMMARY OF THE INVENTION

The present invention provides compounds discovered using computer based virtual ligand screening (VLS) for small molecules that target specific sites on LamR. Using the VLS process and a selected target site, a compound was identified that had the ability to block LamR binding to laminin. Using this compound as a basis for inhibitor discovery, over 40 different compounds with similar chemical structures that behaved similarly in terms of function were identified. The best performing compound disclosed herein displays the ability to block LamR interaction with laminin in vitro as well as functioning as a protein translation inhibitor, growth inhibitor and anti-metastatic agent in tumor cells and murine tumor models.

In one aspect, the present invention provides a pharmaceutical formulation for treating a mammal suffering from a disease mediated by LamR comprising an agent selected from N-(2-hydroxyethyl)dibenzo[a,c]phenazine-11-carboxamide and N-dibenzo[a,c]phenazin-11-yl-4-morpholinecarbothioamide and a pharmaceutically acceptable carrier or diluent.

In another aspect, the present invention provides a method for treating a mammal suffering from a tumor comprising administering to a mammal in need of such treatment an amount of an agent selected from N-(2-hydroxyethyl)dibenzo[a,c]phenazine-11-c arboxamide and N-dibenzo[a,c]phenazin-11-yl-4-morpholinecarbothioamide effective to treat said tumor and a pharmaceutically acceptable carrier or diluent.

In a further aspect, the present invention provides a method for reducing metastatic growth of a tumor in a mammal comprising administering to a mammal harboring a tumor and in need of such treatment an amount of an agent selected from N-(2-hydroxyethyl)dibenzo[a,c]phenazine-11-carboxamide and N-dibenzo[a,c]phenazin-11-yl-4-morpholinecarbothioamide). effective to reduce metastatic growth of said tumor in said mammal and a pharmaceutically acceptable carrier or diluent.

In a still further aspect, the present invention provides a method for treating a patient suffering from Alzheimer's disease comprising administering to a patient in need of such treatment an amount of an agent effective to treat Alzheimer's disease selected from N-(2-hydroxyethyl)dibenzo[a,c]phenazine-11-carboxamide and N-dibenzo[a,c]phenazin-11-yl-4-morpholinecarbothioamide and a pharmaceutically acceptable carrier or diluent.

These and other aspects of the invention will be apparent to those of ordinary skill in the art in view of the present description, claims and drawings.

### BRIEF DESCRIPTION OF THE DRAWINGS

FIGS. 1a-1c—Analysis of LamR structural and sequence features. (a) Sequence alignment between 37LRP/p40 orthologs H. sapiens LamR (residues 1-220) and A. fulgidus S2p ribosomal protein (residues 1-198). Residue numbering is for human LamR. Mutated residues are shaded. Mutations that cause a significant change in laminin binding are in indicated by arrowheads above. Secondary structure for LamR220 is shown according to PROCHECK (70). Light gray lines indicate the N- and C-terminal portions of the structure that are disordered. (b) Superimposition of human LamR220 (darker) and A. fulgidus S2p in ribbon format. α helices and β strands are labeled according to (a). The regions of structural divergence, between the β4-β5 loop (residues 111-118) 5 and after aE (residues 190-194), are labeled. (c) Ribbon diagram of LamR220 in stereo. LamR220 with Phe32, Glu35 and Arg155 labeled and shown in stick (dark), mutations that do not affect laminin-binding are also indicated.

FIGS. 2a and 2b—Analysis of LamR binding affinity for laminin to establish a novel laminin binding site. Binding of LamR and mutants to laminin. LamR220, A. fulgidus S2p, LamR220A 114K/F116A, LamR220 5190 [symbol] P194 polyAla, LamR220Phe32Val, LamR220R155A, LamR220E35K, LamR220F32V/R155A, and LamR220F32V/E35K. n=3+/−SEM. (b) Coomassie blue staining of A. fulgidus S2p, LamR220 and mutants that affect binding for experimental loading control.

FIGS. 3a and 3b-Recombinant wild-type LamR but not laminin binding site mutants inhibits HT1080 cell migration towards laminin (a) Inhibition of cell migration to laminin by wild-type LamR, but not LamR mutants. Laminin alone (grey), laminin+A. fulgidus S2p (horizontal stripes), laminin+wild-type LamR220 (checked), laminin+LamR220F32V/E35K (vertical stripes), laminin+LamR220F32V/R155A (diagonal stripes). n=3+/−SEM. The differences between wild-type LamR220 inhibition and either LamR220F32V/R155A or F32V/E35K are both statistically significant (P<0.0001). (b) HT-1080 cell migration towards FBS is not inhibited by recombinant wild-type or mutant LamR. FBS alone (grey), FBS+A. fulgidus S2p (horizontal stripes), FBS+wild-type LamR220 (checked), FBS+LamR220F32V/E35K (vertical stripes), FBS+LamR220F32V/R155A (diagonal stripes). n=3+/−SEM.

FIG. 4—Identification of a novel target region on the laminin receptor surface. An elaborated druggable region on the LamR protein surface suitable for interaction with inhibitory small molecules (white arrowhead). The residues of which it is composed are shown labeled. The region is shown next to Peptide G residues (flanked by arrows), thought to be important for laminin binding. Surface residues of Peptide G are indicated by the black arrowhead.

FIGS. 5a and 5b—Proposed binding poses of small molecules interacting with LamR identified via virtual ligand screening (VLS). (a) Original hit (MRT/LRI) and (b) best functional analog (MRF/LRI-F/Face). Hydrophobic interactions in black, hydrogen bonds shown by hash marks with bond distances in angstroms. Poses are generated via virtual docking.

FIGS. 6a-6e-Scaffold of active compound chemical structure at various derivative levels. (a) Basic core phenazine molecule with R1-4 substitutions indicating branching or modification points. R5-9 represent tail group elements commonly involved in hydrogen bonding with LamR in the original hit and subsequently discovered analogs (b-c). Increased details of specific chemical structure indicated by performance of the hit compound and analogs. Phenazine core is elaborated to dibenzophenazine (b) and R6/R8 are elaborated into a urea-like (NR₂)₂C═R group (c). R groups in all cases represent substitution or branching points that can be modified to achieve maximum beneficial performance of the compound. (d) Chemical structure of original hit compound MRT (LRI). (e) Chemical structure of most functional analog MRF (LRI-F/Face).

FIG. 7—Full drug target site on LamR inclusive of novel laminin binding site. Partial receptor view. Surface skin represents a novel site important for laminin binding, directly or indirectly. Region targeted by the virtual screen, a potential allosteric effector site, is labeled as Targeted Pocket. The primary amino acids directly comprising the novel laminin binding site are indicated (black arrows)). Uncharacterized linker space between the laminin binding site and the targeted space is shown as white surface (black arrowhead).

FIGS. 8a and 8b—Targeting LamR membrane functions with inhibitors. (a) LamR inhibitors MRT (LRI) and MRF (LRI-F/Face) block recombinant LamR binding to purified laminin coated surface. DBP, dibenzophenazine. (b) Ability of LamR inhibitors to reduce migration of human fibrosarcoma (HT1080) cells to 10% serum. CHX, cycloheximide. *p-value <0.5, **p-value <0.005, n=3.

FIGS. 9a and 9b—Targeting LamR ribosomal and growth functions with inhibitors. (a) Inhibition of cellular translation in several cell lines by LamR inhibitor LRI-F (MRF/Face) by radiolabeled methionine pulse-chase. CHX, cycloheximide (b) Effects of treating HT1080 cells with LamR inhibitors. Dose dependent growth effects occur. *p-value <0.0001, n=3.

FIGS. 10a-10d—Effects of LamR inhibitors on experimental metastasis of HT1080 cells. in vivo imaging of intravenously introduced luciferase carrying HT1080FLuc cells implanted into the lungs after (a) pre-treatment of cells with LamR inhibitor LRI-F prior to injection or (c) treatment 2 hours following intravenous injection of cells. (b) Survival data for pre-treatment experimental metastasis model shown in (a). (d) Quantitation of lung tumor signal with and without treatment for the experiment shown in (c).

### DETAILED DESCRIPTION OF THE INVENTION

In order to identify regions of LamR that are important in its multiple functions and to then target those regions, computer-assisted modeling was used to rapidly screen millions of drug-like compounds. Pursuant to the present invention, discovery of the correct type of small molecule has allowed for the specific inhibition of LamR, which will make for an effective therapy against several pathologies such as cancer, certain viral infections, neurodegenerative diseases (including prion diseases and Alzheimer's Disease) as well as in bacterial infections. Virtual ligand screening (VLS) (68-71) requires a discrete region of a receptor to be targeted; screening of the entire receptor is too complex to be accurately modeled. Typically a region of known importance is selected for targeting but, in the instant invention, in the absence of any known ligand binding positions, a region for initial targeting was chosen based on its proximity to a reported laminin binding site (Peptide-G, amino acids 161-180). This region was simultaneously identified as the largest region of “druggability” based upon predictive modeling of protein structure using MolSoft ICM software “icmPocketFinder”(MolSoft). The region of “druggability” in this context is defined as a pocket or space that can potentially accommodate ligands, usually a partially recessed and non-loop region of the receptor.

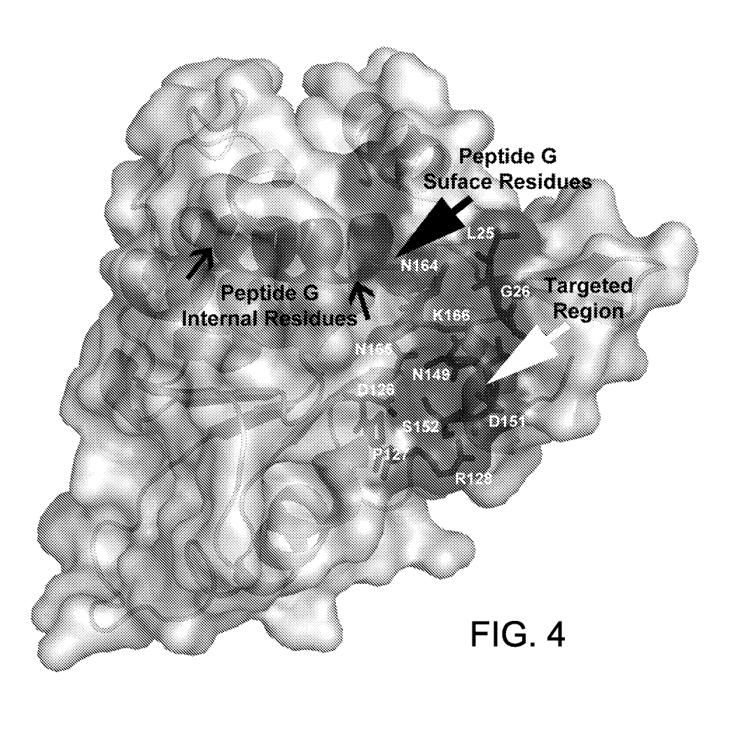

Based on these factors, the region ultimately chosen represented a novel site previously un-described in both functional importance to LamR and susceptibility to small molecules. The region is comprised primarily of amino acid residues L25-G27, D126-R128, N149-S152, N164-K166 and the surface area they encompass (FIG. 4). Of these residues, only N164-K166 are present in Peptide G. This region has not previously been reported to have importance for LamR function. The source for this numbering system is the full length LamR sequence (46, PDB 3BCH). It is as in the PDB entry for the LamR structure as well as in GenBank. NCBI Protein accession: NP_{—}001012321.

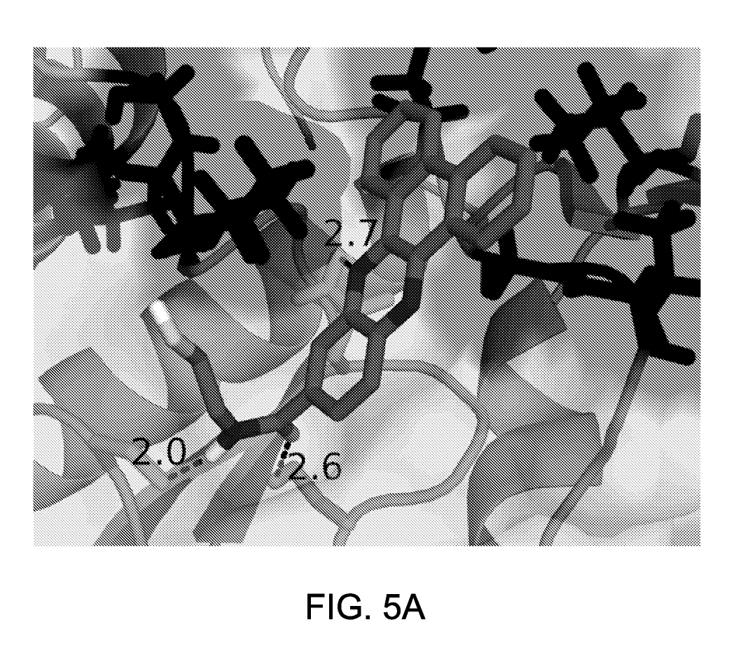

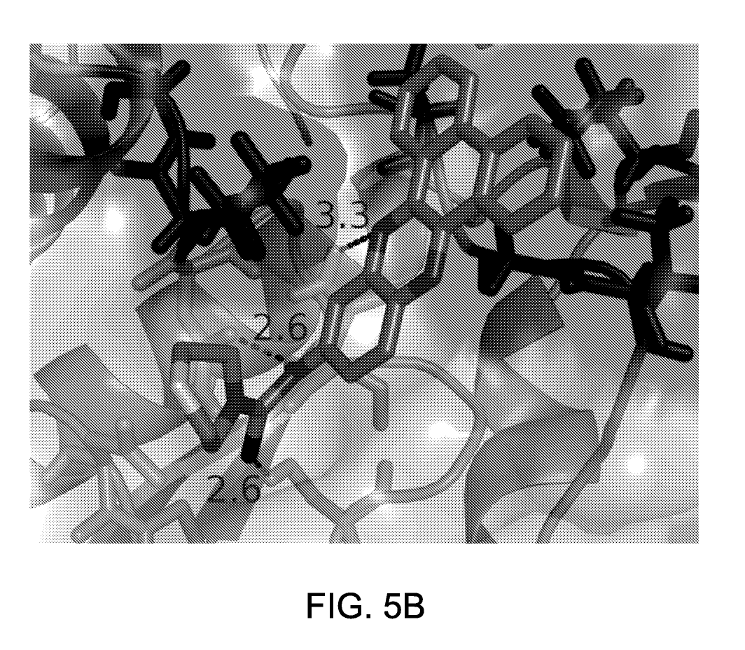

Targeting this pocket allowed the screening process to identify a single compound that had the desired properties consistent with an inhibitor of the laminin receptor, both biochemically tested in vitro and in the cellular context. The compound partially fits within the region, interacting with the receptor via three distinct hydrogen bonds and numerous hydrophobic contacts (FIG. 5a). Subsequent analogs of the compound, which were discovered by searching databases of publically available chemicals (ChemBridge, ChemDiv, Enamine and PubChem), interacted with the receptor in a similar manner when virtually docked to LamR (FIG. 5b). These analogs behaved successfully in the assays described herein, some performing equally to the original hit and others representing a significant improvement in the desired properties of potency and solubility. The most successful compounds interacted with the receptor primarily via a nitrogen hetero-atom within the phenazine core of the molecule, the electron accepting oxygen or sulfur of a urea or thiourea group and the phenazine-side nitrogen of the same urea/thiourea group. Other compounds that do not contain urea/thiourea groups are successful provided they maintain the same atomic characteristics at similar positions and bond distances as to maintain its interaction with the receptor. A chemical synthesis scheme for obtaining these compounds is available from ChemBridge for both the initial hit (5a, MRT) and the analog (5b, MRF). Synthesis of MRF is described below in Example 4. FIG. 5a shows the initial compound, MRT/LRI N-(2-hydroxyethyl)dibenzo[a,c]phenazine-11-carboxamide bound to LamR in the target pocket described in FIG. 4. FIG. 5b shows the analog, MRF/LRI-F/F ace (N-dibenzo[a,c]phenazin-11-yl-4-morpholinecarbothioamide) which is preferred for use in the present invention.

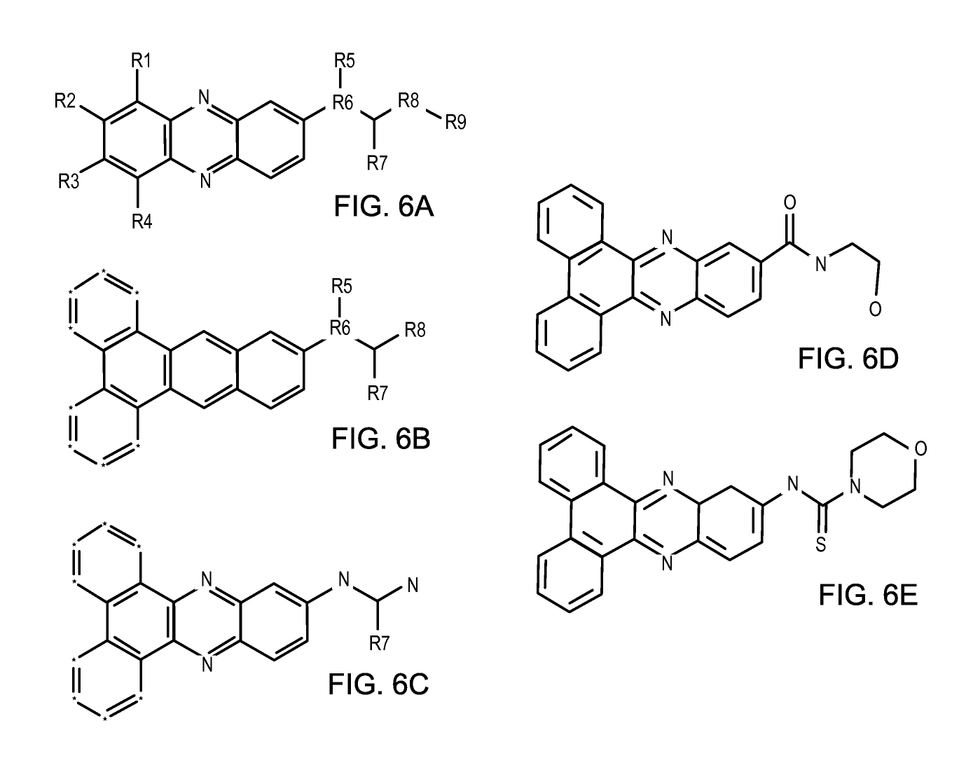

FIG. 6a shows the basic core phenazine molecule with R1-R4 substitutions left open for modification. R5-R9 represent a tail group wherein the R5 and R7 positions are commonly involved in hydrogen bonds with the receptor either as an acceptor or donor. R6 and R8 as well as the linker are commonly represented in functional analogs as nitrogen or carbon. R9 is typically a connector to a larger tail group that affects solubility more than potency. FIG. 6b shows the elaborated core featuring a dibenzo-group in addition to a phenazine core. * represents any substitution into the ring structure that may affect its properties. FIG. 6C shows the working lead scaffold featuring dibenzo-groups and a urea group specifics. R7 is typically represented in functional analogs as oxygen or sulfur.

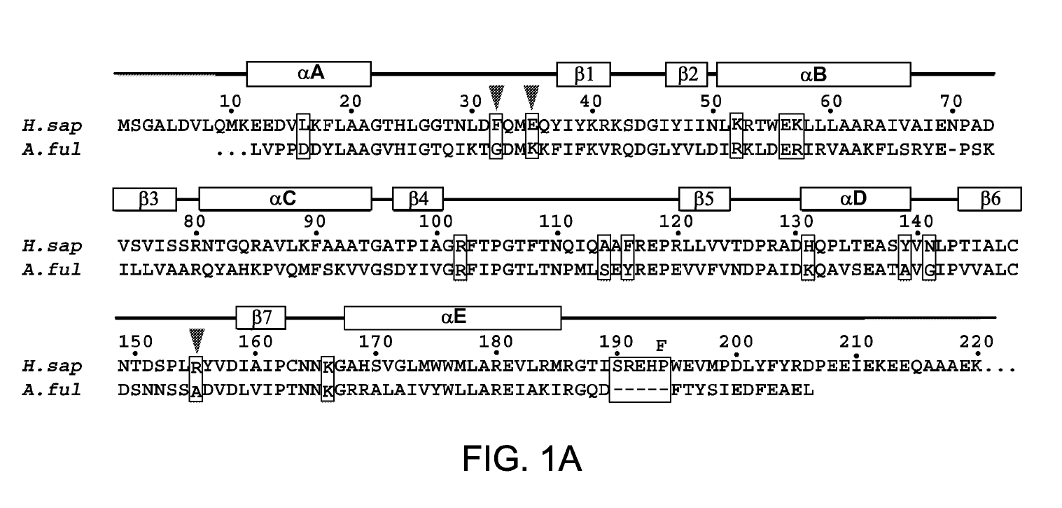

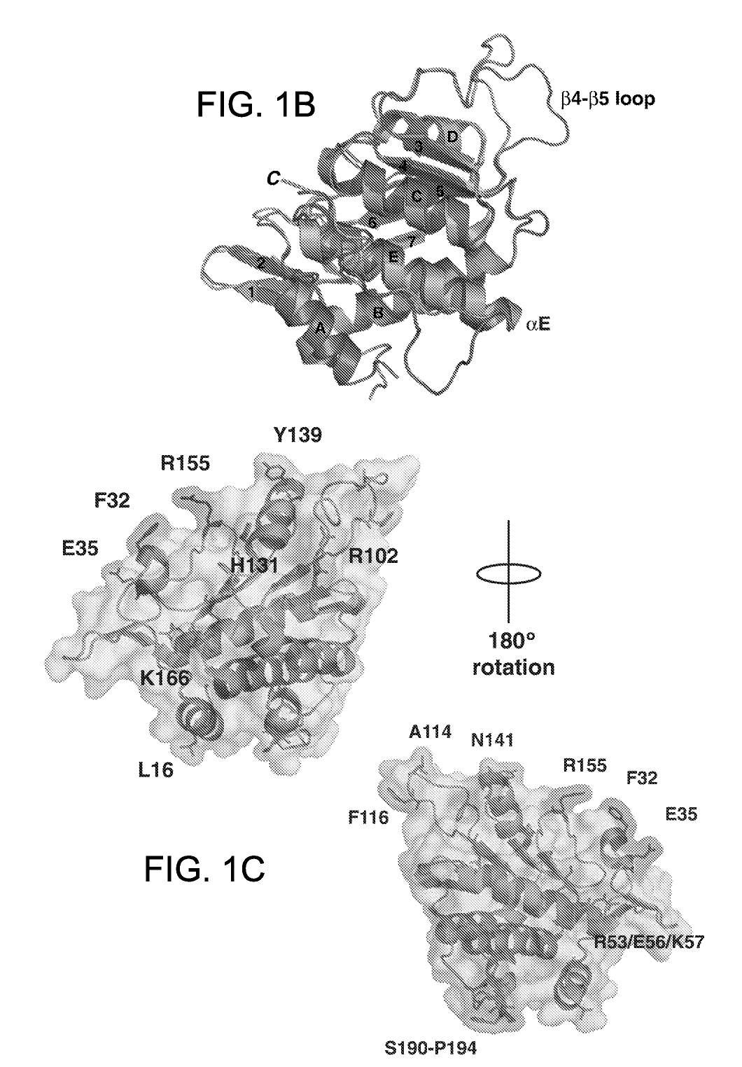

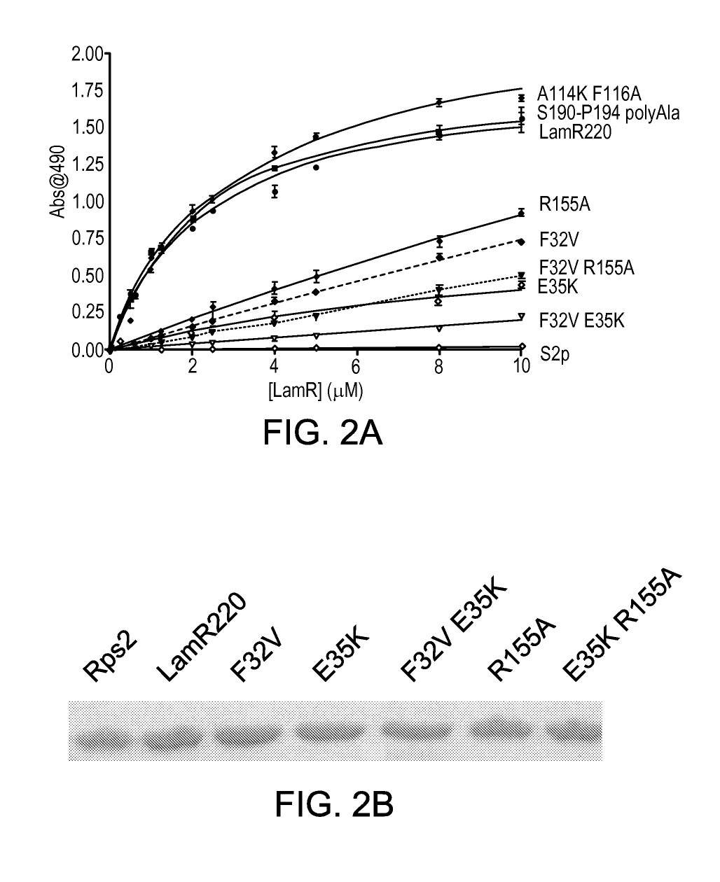

Although Peptide G has been reported to bind to laminin, the majority of the residues that comprise the sequence are not surface exposed and therefore were not discretely targeted by the screen. The pocket composed of the region shown in FIG. 4, near a novel laminin binding site (FIGS. 1 and 2), may be an important allosteric site, as it has not been reported to function directly in laminin binding.

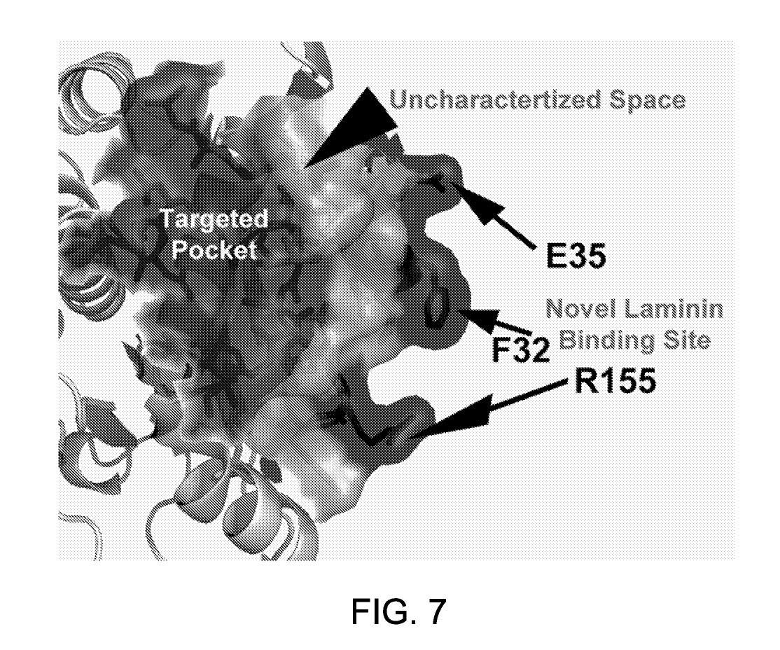

Of the many mutations tested and screened for abnormal laminin binding to LamR, F32, E35 and R155 stood out as severely affecting the ability to bind laminin (FIG. 2). These three amino acid residues are within close structural proximity to each other and may form a core laminin binding region. Without wishing to be bound by theory, it is believed that this site (F32, E35, R155) is part of a larger, full binding site that stretches to the pocket described above (FIG. 4), as both sites are within structural proximity to one another (FIG. 7). This full site would consist of amino acids L25-E35, V124-R128, L134, L146, N149-S152, R155 and N164-K166 and the region they encompass. FIG. 7 depicts the originally targeted pocket in dark shading and white label (L25-G27, D126-R128, N149-S152, N164-K166), the novel laminin binding site (F32, E35, R155) indicated by arrows and uncharacterized connecting space indicated by black arrowhead (T28-E35, V124, L134, A146, P153-Y156).

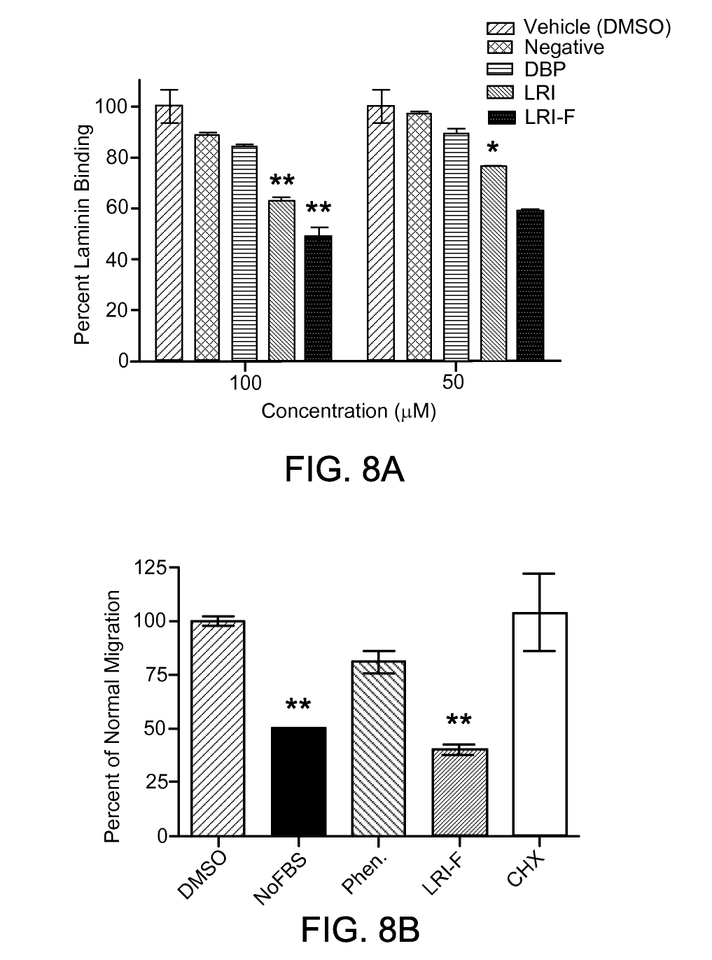

Compounds that interact with this described pocket, MRT (LRI) and MRF (LRI-F), display the properties of LamR inhibitors and are preferred for use in the methods of the present application. FIG. 8a shows the dose-dependent inhibition of laminin binding by LamR in an in vitro assay that uses purified recombinant protein to quantitate the level of interaction. Treatment of LamR with the aforementioned inhibitors reduced binding approximately 50 percent relative to the vehicle control (DMSO alone). Binding was not reduced by several negative control compounds (Negative and DBP).

In order to understand how this in vitro result translated into the cellular context, the ability of cells to migrate toward growth medium was tested in the presence and absence of the LamR inhibitors of the present invention. Since LamR on the cell surface serves the purpose of binding extracellular matrix laminin and facilitates migration and metastasis, it was expected that as inhibitors of cell surface LamR, the compounds of the present invention would reduce the migratory capability of cells. FIG. 8b shows this inhibition of migration upon treatment of cells with the LamR inhibitors. The migration of HT1080 cells was reduced to a level equivalent to the no migration control of no serum (No FBS). This effect was not seen for the vehicle alone (DMSO) or a negative control compound (Phen.) and furthermore the effect was not due to translational inhibition of the compound, as translational inhibition by cyclohexamide (CHX) did not result in loss of migratory ability.

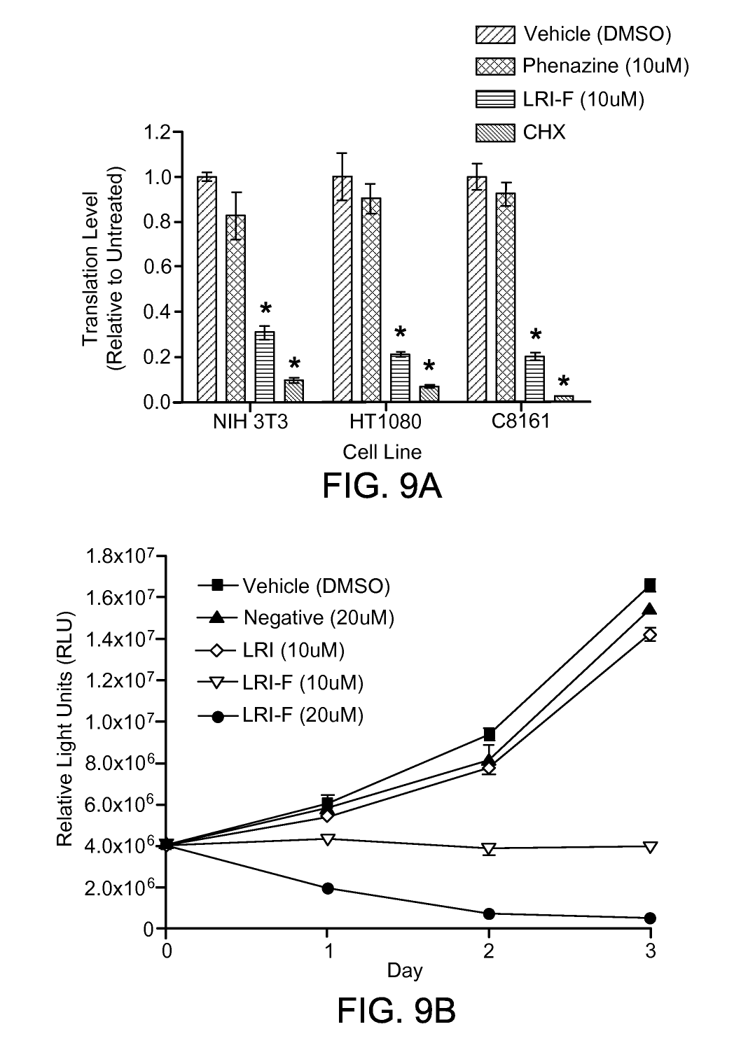

In addition to its membrane functions of laminin binding and migration, LamR acts as a ribosomal protein that has been shown to be essential for synthesis of proteins—the process of translation. As expected for compounds with these properties, the LamR inhibitors of the invention attenuated the translation process. A panel of cell lines (3T3, HT1080 and C8161) treated with 10 μM of LRI-F (MRF/Face) displayed a dramatic reduction in translation of newly synthesized proteins relative to an untreated control as assayed using a ³⁵S-Methionine pulse-chase method. Vehicle and a negative control (Phen.) resulted in no such inhibition (FIG. 9a).

LamR has been shown to be essential for the growth and viability of cells, another of its several functions. The analog compound LRI-F, an inhibitor of LamR, affected these functions. The compound induced growth inhibitory effects in HT1080 cells in a dose-dependent manner (FIG. 9b), demonstrating that it has antitumor activity.

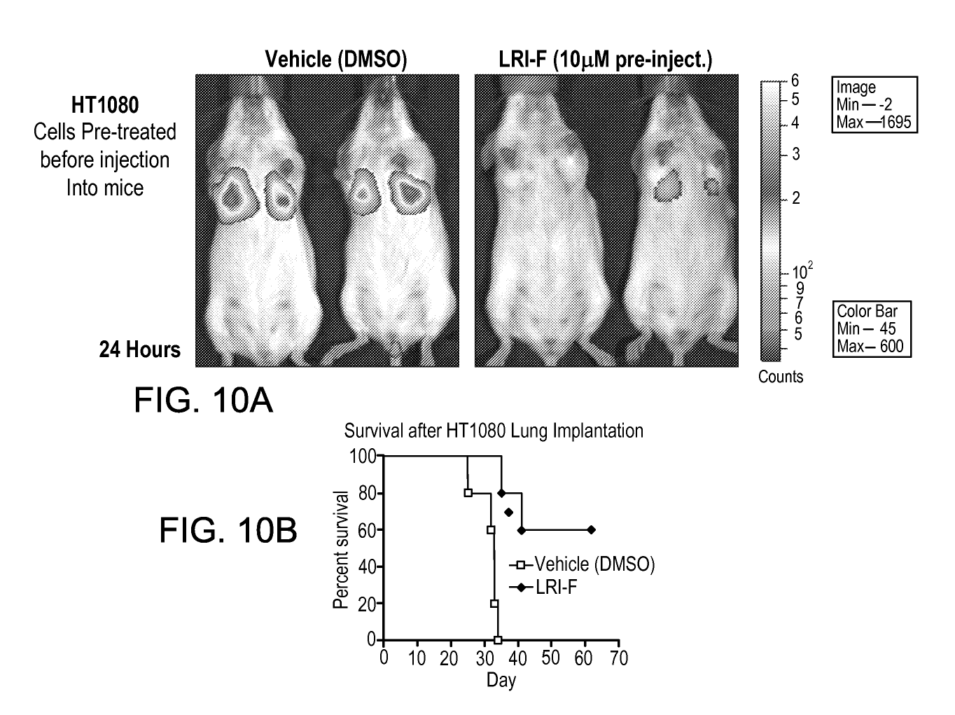

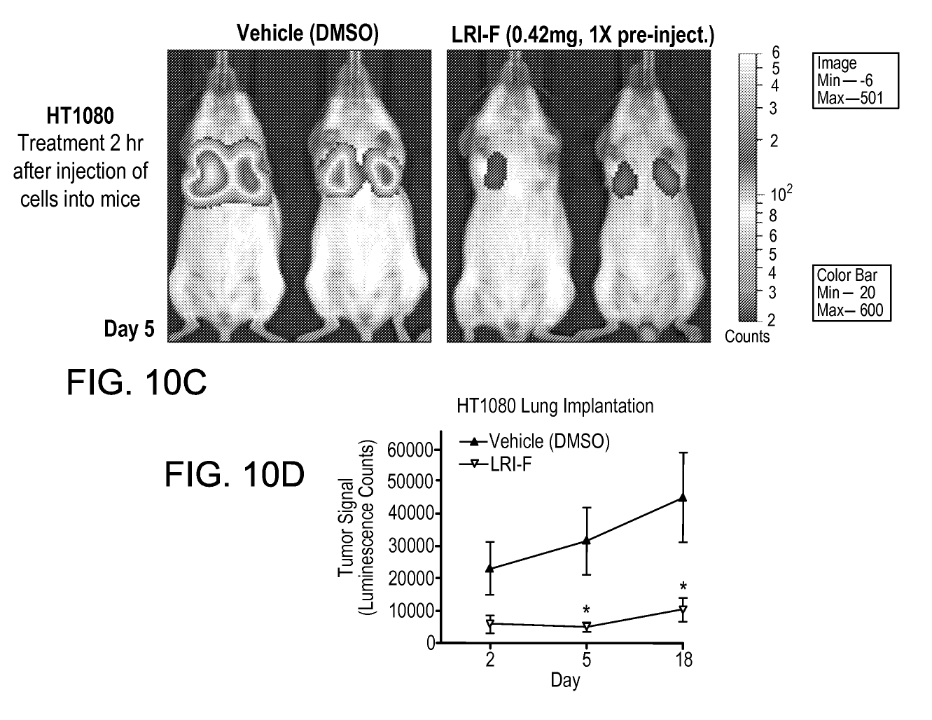

In addition, LamR has been described to be a critical factor of tumor metastasis. Clinical uses of the LamR inhibitors disclosed herein include their use as anti-metastatic anti-tumor agents. In a manner similar to that observed for the above-described in vitro cellular migration assays, compound LRI-F has such anti-metastatic properties in a murine model of experimental metastasis. Pretreatment of HT1080 cells with 10 μM of LRI-F (FIG. 10a) resulted in a marked decrease of the cells' ability to implant into the lungs of SCID mice after intravenous injection via the tail vein. This decreased the colonization ability of the tumor cells and resulted in prolonged survival of the mice compared to cells pretreated with the DMSO vehicle alone (FIG. 10b). In a similar experiment, mice that received LRI-F treatment (0.42 mg/20 g injected subcutaneously once 2 hours post-injection of the cells) displayed a decreased tumor signal from the lungs, indicating that a fraction of the cells were prevented from implanting by the compound. This effect is shown in FIG. 10c-d. Tumor cells were imaged via their expression of an exogenous firefly luciferase gene using the IVIS in vivo imaging system (Caliper/Xenogen).

Pursuant to the present invention, “treating” a mammal suffering from a tumor encompasses inhibiting or reducing the amount of metastasis of a tumor. Eliminating or reducing a tumor cells' ability to metastasize will be especially important when used in conjunction with surgery where there is a high risk of releasing metastatic cancer cells into the bloodstream or lymphatic system. In addition, the compounds of the invention can be used in conjunction with other anti-tumor treatments such as surgery, radiation and chemotherapy, leading to the total elimination of tumor cells from the body.

In an alternate embodiment of the present invention, the agents described above can be used to treat patients suffering from Alzheimer's Disease. It has been reported that LamR is involved with prions, which are related to Creutzfeldt-Jakob disease in humans (75). Creutzfeldt-Jakob disease is a debilitating and fatal neurodegenerative disease believed to result from the progressive death of brain cells caused by the accumulation of prions. Prion buildup is somewhat similar to the deposition of ABeta plaques in the brains of Alzheimer's patients. Numerous links connecting LamR to Alzheimer's Disease have appeared in the scientific literature. For example, it has been shown that laminin, the normal binding partner of LamR, interacts with ABeta (73). This interaction was also shown to prevent fibrillogenesis of ABeta (72), one of the key events in the formation of ABeta plaques. Interestingly and importantly, laminin partially blocks the toxicity that ABeta has towards neuronal cells (74). In addition, LamR has been reported to be irregularly modified in a mouse model of Alzheimer's Disease, such that it may no longer be able to bind its substrates (76). Without wishing to be bound by theory, it is believed that targeting LamR with the compounds disclosed herein will lead to a neuroprotective effect, reducing or eliminating the toxicity of the ABeta peptides and plaques to the brain's neurons. LamR is one of potentially many receptors that mediate the cytotoxic effects of ABeta in cells, which can include the triggering of apoptosis (programmed cell death), mitochondrial defects that are prohibitory to cell growth and have a role in tauopathy, another prominent aspect of Alzheimer's Disease. By inhibiting the abnormal LamR signaling, blocking the harmful effects of Abeta will be possible. Paper Example 1 below describes an animal model of Alzheimer.s Disease in which the effects of the compounds of the present invention can be tested.

In a preferred embodiment of the invention the mammal is suffering from a tumor, in which the cells of the tumor express greater levels of LamR compared to normal cells of the same lineage and the compounds are administered systemically. The different levels of LamR result in target-mediated delivery, i.e., preferential binding of the compounds of the present invention. After administration of the compounds of the present invention, tumor cells which overexpress LamR will “soak” a greater number of compound particles per cell—thus delivering an effective antitumor dose.

This treatment is not limited to cells that over-express LamR, but since most tumors over-express LamR it will likely work better in such cells. Tumor cells often are more sensitive to drug activity in general, because they are growing, duplicating and metabolizing at a considerably faster rate than non-transformed tissue, which is commonly senescent.

“Greater levels” of expression generally refer herein to levels that are expressed by tumor cells (as compared to non-tumor cells) and result in such preferential binding, e.g., at least a 3-fold greater binding, preferably at least a 30-fold greater binding and, most preferably at least a 300-fold greater binding. The increased level of expression in tumor cells can be evaluated on an absolute scale, i.e., relative to any other LamR expressing non-tumor cells described, or on a relative scale, i.e., relative to the level expressed by untransformed cells in the same lineage as the transformed cancer cells (e.g., melanocytes in the case of melanoma; hepatocytes in the case of hepatic carcinoma; ovarian endothelial cells in the case of ovarian adenocarcinoma, renal endothelial or epithelial cells in the case of renal carcinoma).

A subject to be treated by the methods of the present invention is a mammal and preferably a human.

As used herein, the term “tumor” refers to a malignant tissue comprising transformed cells that grow uncontrollably. Tumors include leukemias, lymphomas, myelomas, plasmacytomas, and the like; and preferably solid tumors. Examples of solid tumors that can be treated according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, epidermoid carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, neuroglioma, and retinoblastoma. As noted above, the method of the invention depends on expression of LAMRs by cells of the tumor targeted for treatment.

The term “about” or “approximately” usually means within an acceptable error range for the type of value and method of measurement. For example, it can mean within 20%, more preferably within 10%, and most preferably still within 5% of a given value or range. Alternatively, especially in biological systems, the term “about” means within about a log (i.e., an order of magnitude) preferably within a factor of two of a given value.

The term “therapeutically effective” when applied to a dose or an amount refers to that quantity of a compound or pharmaceutical composition that is sufficient to result in a desired activity upon administration to a mammal in need thereof. As used herein with respect to the compounds of the invention, the term “therapeutically effective amount/dose” refers to the amount/dose of a compound or pharmaceutical composition containing the compound that is sufficient to produce an effective anti-tumor response upon administration to a mammal

The dose to be administered (“the effective amount”), can be determined by escalating the dose from a minimum level to an effective concentration. Such dosage adjustments are well known to those of ordinary skill I the art. Knowledge of a dose at which signs of toxicity begin to show may be determined in a similar fashion. The minimum effective dose, determined by titration and monitoring, is preferred as a therapeutic dose, determined experimentally in murine models and in approved clinical trials for human usage. In the current invention, the dose selected for treatment of mice was the maximum deliverable amount due to solubility constraints of the compound described (0.42 mg/20 g). Solvation in this case was achieved in DMEM+10% FBS as described. At this dose, the current maximum available, there is no apparent toxicity to mice for a continued timeline of treatment exceeding 3 weeks.

The present invention includes pharmaceutical formulations or dosage forms for treating mammals suffering from diseases mediated by LamR disclosed herein. When formulated in a pharmaceutical composition, of the compounds of the present invention can be admixed with a pharmaceutically acceptable carrier or excipient. The phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicles with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Alternatively, the carrier can be a solid dosage form carrier, including but not limited to one or more of a binder (for compressed pills), an encapsulating agent, a flavorant, and a colorant. Suitable pharmaceutical carriers are described in “Remington's Pharmaceutical Sciences” by E. W. Martin.

The preferred route of administration of the compounds of the present invention is parenteral and most preferably systemic. This includes, but is not limited to intravenous, intraperitoneal, intra-arteriole, intra-muscular, intradermal, subcutaneous, intranasal and oral. These routes of administration will permit homing of the compounds to tumor cells wherein, only tumor cells which express higher levels of the laminin than normal cells of the same lineage are targeted.

It has been demonstrated herein that the general region described in FIG. 7 has therapeutic importance both in terms of laminin binding and susceptibility to small molecules.

The present invention is described below in examples that are intended to further describe the invention without limiting the scope thereof. In the experiments described above and in the examples below, the following materials and methods were used:

Recombinant LamR and A. Fulgidus S2p Expression and Purification.

Residues 1-220 of human 37 kDa LamR precursor protein (LamR220) were subcloned from full-length LamR cDNA into an E. coli expression vector that includes a TEV-cleavable, N-terminal 6×His-tag. The construct was verified by automated DNA sequencing. The vector encoding LamR220 was transformed into E. coli strain BL21 (DE3*), and cultures were grown in Luria broth media at 37° C. to an OD600 of 0.6. Protein expression was induced by the addition of isopropyl-thiogalactopyranoside (IPTG, 0.1 mM) for 16 hr at 20° C. Cells were harvested, resuspended in lysis buffer (50 mM Tris (pH 8.0), 300 mM NaCl, 0.1% Triton X-100, 10% glycerol, EDTA-free protease inhibitor tablet (Roche)) and lysed by French press. The lysate was centrifuged at 16,000 RPM for 30 min and the supernatant was collected. The soluble fraction was purified by Ni-NTA chromatography (Qiagen), followed by gel filtration chromatography (Superdex 75, Amersham). Protein was concentrated in spin concentrators (Amicon, Millipore). Residues 1-208 of A. fulgidus S2p were cloned, expressed and purified utilizing the same conditions as human LamR220, as was full-length LamR (1-295).

LamR Site-Directed Mutagenesis.

The LamR plasmid constructs were used as the backbone for generating LamR point mutants in the LamR coding region with the QuikChange site-directed mutagenesis kit XL II (Stratagene). Complementary forward and reverse primers were designed to mutate LamR residues. PCR-based point mutations were made using thermal cycling according to the manufacturer (Stratagene) protocol, the PCR product was digested with DpnI restriction enzyme (NE Biolabs) and transformed into E. coli strain XLII Blue (Stratagene). LamR vectors positive for mutation were verified by automated DNA sequencing and LamR mutants were transformed into E. coli strain BL21 (DE3*). Protein expression and purification was carried out using the same conditions for wild type LamR and the A. fulgidus S2p ribosomal protein.

In Vitro Binding Affinity for Laminin.

Wild type and mutant LamR binding affinity for laminin were tested in vitro. Clear polystyrene ELISA 96-well microplates, pre-coated with murine laminin (NEBiolabs), were blocked for 1 hour at 37° C. with blocking buffer (2% FCS, 1 mg/ml BSA, 0.1% sodium azide in PBS). Wells were incubated with increasing concentrations of purified wild type or mutant LamR for 1 h at 37° C. If a small molecule's effects on laminin binding were to be determined, the small molecule was pre-incubated with purified LamR for 15 minutes at 37° C. prior to addition of the mixture to the pre-coated plate. Each well was washed three times with wash buffer (0.5% Tween in PBS). Penta-His HRP conjugate (1:500) (Qiagen) was incubated for 2 h at RT and wells were washed three times with wash buffer. Substrate solution was added and incubated for approximately 10 minutes before reading the fluorescent absorbance detected at 490 nm on an ELISA plate reader (ELX800, Biotek Instruments, Inc.). Controls for buffer alone and binding of a nonspecific protein, A. fulgidus S2p, were also tested. Experiments were performed in triplicate. A binding curve and K_{d} were generated for wild type and mutant LamR using Prism software. The K_{d} was calculated using a one-site binding hyperbola and the equation Y=Bmax×X/(K_{d}+X). Each group was tested in triplicate and binding affinity was determined by normalizing to background fluorescence.

In Vitro Cellular Migration.

HT1080 cells were tested for their ability to migrate toward normal growth media supplemented with 10% FBS or to 10 μg/mL purified laminin (Invitrogen) using the CytoSelect™ 24-well cell migration assay (8 μm, Colorimetric Format) (Cell Biolabs, Inc.). HT1080 cells were obtained from the American Type Culture Collection (Manassas, Va.). HT1080 cells were maintained in Dulbecco's modified Eagle's medium—4.5 g/ml glucose with 10% FBS. All basal media were supplemented with 100 μg/ml penicillin-streptomycin and 0.5 μg/ml amphotericin B (all from Mediatech). Briefly, 1.5×10⁵ cells in unsupplemented medium were added to the upper insert chamber of each well. The lower reservoir contained either 500 μl of unsupplemented medium or medium supplemented with 10% FBS. In the case of assaying migration to laminin and the effects of LamR mutants, the lower reservoir included 500 μl of unsupplemented medium, purified laminin, or purified laminin and recombinant wild-type LamR, mutant LamR, or A. fulgidus S2p. For other migration tests, the lower reservoir contained 500 μl of unsupplemented medium, medium supplemented with 10% FBS, or 10% FBS and recombinant wild-type LamR, mutant LamR, or A. fulgidus S2p. The final concentration of all proteins was 1 μM. For measuring the effects small molecules had on the migration of cells, cells in culture were pre-treated for 2 hours with 5-10 μM of small molecule prior to addition to the upper chamber. To control for possible translational inhibition effects on migration, 50 μg/mL cycloheximide was added 3 hours prior to assessment as a separate sample. After incubation at 37° C. for 2-5 h, the upper membrane of each insert was thoroughly washed three times with dH2O to remove nonmigratory cells and then incubated in cell stain solution for 20 min at room temperature. Inserts were washed again in dH2O and then air-dried. After agitation for 10 min at room temperature in extraction solution, 100 μl of each sample was transferred to an ELISA plate and read at 630 nm. Assays were performed in triplicate. Results were plotted using GraphPad Prism software, and statistical significance was calculated using a standard Student's t-test.

Assessing Cellular Translation with Pulse-Chase Radiolabeling.

To label newly synthesized proteins, cells were pulsed with 20 μCi/mL ³⁵S-methionine radiolabeled amino acid (PerkinElmer) in standard growth media lacking methionine (MP Biomedicals) for 2 hours at 37° C. Following the pulse, normal growth medium was added for the chase to restore normal conditions for 90 minutes. MPER mammalian protein extraction reagent (Pierce) was used to lyse cells and collect protein. To quantity the amount of newly synthesized protein containing the radioisotope, 20 μg total protein was precipitated with 10% TCA for 20 minutes at 4° C. Precipitant was vacuum filtered and washed on glass microfiber filters (Whatman) using 5% TCA followed by 95% ethanol. Filters were dried under a heat lamp prior to addition of 3 mL scintillation fluid to count radioisotope incorporation by events on a Beckman LS3801 scintillation counter (Beckman). For measuring the effects small molecules had on the translation, cells in culture were pre-treated for 18 hours with 1-10 μM of small molecule prior to pulse/chase. As a control for arresting translation, 50 μg/mL cycloheximide was added 3 hours prior to assessment.

Inhibition of Experimental Metastasis by LamR Inhibitors.

Firefly luciferase expressing HT1080 human fibrosarcoma cells were either pre-treated for 15 minutes with 10 uM of experimental inhibitory compounds or equivalent volume of vehicle (DMSO) or treated with 0.42 mg/20 g in (DMEM+10% FBS) or vehicle intra-peritoneal (i.p.) 2 hours following injection of cells into mice. Cells were washed once in PBS and re-suspended to a concentration of 1×10⁶ cells/mL in PBS. 0.5 cc of the cell suspension (500,000 cells) was injected intravenously via the tail vein into SCID mice. Tumor implantation was monitored with IVIS Spectrum imaging technology (Caliper LS/Xenogen) following intra-peritoneal injection of luciferin substrate. Briefly, a camera is used to detect bioluminescent signal emitted from luciferase expressing cells. Mice are injected i.p. with 0.3 cc of 15 mg/mL beetle luciferin substrate (Promega) in PBS. Mice are anesthetized with isofluoran (2%)/oxygen mixture prior to imaging 5 minutes post substrate injection. Light is collected over a 1 minute imaging period at high binning to detect lung signal. Living Image Software version 3.0 (Caliper LS) is used to quantify and represent light signal.

### Example 1

### Identifying a Novel Laminin Binding Site on LamR

The interaction between LamR and laminin mediates changes in the cell environment that affect cell adhesion and tumor growth and metastasis (6, 7, 23, 59-62). To better understand LamR interactions with laminin, LamR mutants were designed based upon analysis of sequence conservation and the crystal structures of human LamR220 (PDB code 3BCH) and its non-laminin binding ortholog, Archaeoglobus fulgidus S2 ribosomal protein (PDB code 1VI6). In total, 14 mutants were analyzed individually (FIG. 1-2). Protein expression and purification of each mutant, including by gel filtration, was similar to that of wild-type LamR220, which indicated that the mutations did not interfere with protein folding or stability (FIG. 2).

Only mutations Phe32, Glu35, and Arg155 resulted in an appreciable loss of binding affinity (FIG. 2). Single point mutation of LamR Phe32 to Val (the corresponding residue in S. cerevisiae S2 ribosomal protein), or Glu35 to Lys, or Arg155 to Ala (the corresponding residues in A. fulgidus S2 ribosomal protein) resulted in a decrease in laminin binding (8- to 42-fold increase in K_{d}). Double point mutation of Phe32 and Glu35 (abbreviated F32V/E35K) or Phe32 and Arg155 (abbreviated F32V/R155A) resulted in a greater loss of laminin binding activity than the single mutants. These data demonstrate that Phe32, Glu35, and Arg155 comprise a primary laminin binding site. These residues are conserved among mammalian laminin-binding species, but not in A. fulgidus S2 ribosomal protein ortholog, which does not bind laminin.

Interestingly, mutation of two regions of structural divergence between human LamR and A. fulgidus S2p, the β4-β5 linker and a five residue insertion after αE, did not affect laminin binding, suggesting that they contribute to other LamR functions such as membrane attachment. In addition, mutation of Lys 166 to Ala, which was previously implicated in laminin binding (47), did not affect binding in vitro.

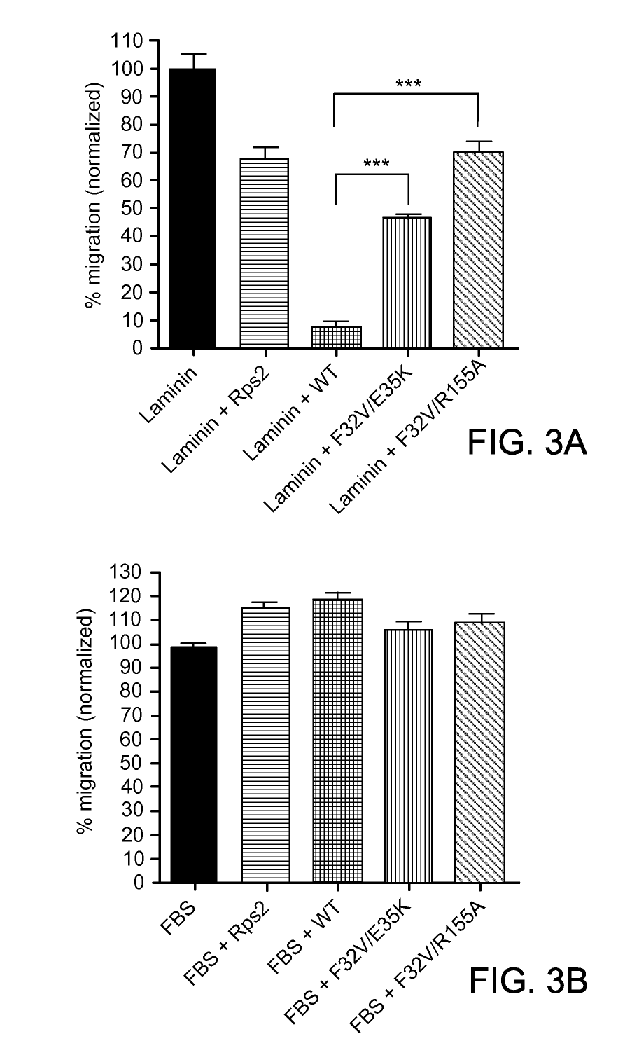

To assess the physiological significance of residues Phe32, Glu35, and Arg155 in laminin binding a cellular assay was developed to test wild-type and mutant LamR function. Purified recombinant LamR was utilized as a soluble decoy to interfere with endogenous LamR cell surface interactions with laminin in a cell migration assay. The ability of recombinant wild-type or mutant LamR220 to inhibit HT1080 cell migration towards laminin was examined in a Boyden chamber in which cells were added to the top chamber and laminin or laminin with recombinant protein were added to the bottom chamber. When recombinant wild-type LamR220 was added with laminin, HT1080 cell migration towards laminin was 8% of the migration observed relative to migration to laminin alone (FIG. 3a, WT). These data show that recombinant LamR220 interacts with laminin in solution, inhibiting cellular migration towards laminin. Cellular migration to recombinant protein alone was not observed. Protein purity and equal loading was examined by SDS-PAGE. A. fulgidus S2p, which does not exhibit laminin binding activity in vitro, was utilized as a negative control for migration inhibition (FIG. 3a, Rps2). LamR220 mutants F32V/E35K and F32V/R155A, which exhibited loss of laminin binding in vitro, were examined for loss of inhibition and restoration of cell migration to laminin. Both LamR220 mutants F32V/E35K and F32V/R155A demonstrated a significant loss of inhibition of cell migration to laminin (FIG. 3a, F32V/E35K and F32V/R155A). LamR220 F32V/R155A behaved similarly to A. fulgidus S2p (negative control), with 70% migration observed compared to migration to laminin alone. LamR220F32V/E35K resulted in 46% of migration compared to migration to laminin alone. Cell migration towards 10% FBS was not inhibited by wild-type LamR220, mutant LamR220, or A. fulgidus S2p, which suggests that inhibition of cell migration to laminin by wild-type LamR220 is specific to interactions between LamR and laminin (FIG. 3b). Together, these data demonstrate the physiological role of Phe32, Glu35, and Arg155 in laminin binding.

In the setting of cancer, interactions between LamR and laminin contribute to modifications of the extracellular matrix structure that affect cancer cell growth and proliferation and tumor invasion and metastasis, and activate proteolytic enzymes and their regulators (7, 63-66). The specific inhibition of LamR interactions with laminin has led to the discovery of compounds useful for the prevention of tumor growth and metastasis

### Example 2

### Expression and Characterization of LamR Mutants

The crystal structure of a biologically active domain of human LamR, LamR220 has been previously resolved (46). LamR220 binds laminin with similar affinity to full-length LamR (LamR295)(46), demonstrating that LamR220 is sufficient for laminin-binding. For these studies, LamR220 was used for mutagenesis due to its higher level of expression and purity compared to LamR295. Despite the implication of residues 161-180 in laminin binding(47), the laminin [symbol] binding site has remained elusive since the crystal structure of human LamR revealed that a large portion of this domain is not solvent accessible. To better understand LamR interactions with laminin, LamR mutants were designed based upon analysis of both sequence conservation and the crystal structures of human LamR220(46) (PDB code 3BCH) and its non-laminin binding ortholog, Archaeoglobus fulgidus S2 ribosomal protein(50) (PDB code 1VI6).

In total, 14 mutants were analyzed individually (Table 1, FIG. 1a). Protein expression and purification of each mutant was similar to that of wild-type LamR220, which indicated that the mutations did not interfere with protein stability or folding. The purity of the proteins was monitored by SDS [symbol] PAGE. All mutants were examined for their ability to bind immobilized laminin. Purified recombinant LamR220 (greater than 95% purity) was incubated in serial dilution on pre-coated laminin plates, and binding was detected by anti-His HRP. LamR220 specifically binds laminin in vitro and does not bind fibronectin, another component of the extracellular matrix (data not shown). As a control, laminin binding for A. fulgidus S2p ribosomal protein was tested, which should not interact with laminin. A. fulgidus S2p does not exhibit laminin-binding activity, as expected (FIG. 2a). These data support the hypothesis that differences between human LamR and A. fulgidus S2p are important for laminin binding.

### Example 3

### Mapping of the Laminin-Binding Region of LamR by Site-Directed Mutagenesis

Structural and sequence analyses afforded selection of solvent-exposed residues that could contribute to laminin binding. The studies reported on herein examined the role of three categories of mutations: structurally divergent regions between human LamR and A. fulgidus S2p, residues previously implicated in laminin binding, and non-conserved residues between human LamR and A. fulgidus (FIG. 1a). Human LamR and its ortholog A. fulgidus S2p ribosomal protein share 32% sequence identity. Superimposition of the structures of LamR220 and A. fulgidus S2p revealed two areas in which the structures are divergent (FIG. 1b): a segment between P4 and β5 (residues 111-118 in LamR) and a segment after the last a helix (aE) (residues 190-194 in LamR), in which LamR contains a five-residue insertion relative to A. fulgidus S2p. In A. fulgidus S2p, the segment between β4 and β5 (residues 111-118 in LamR), is stabilized in a folded-back conformation via a salt bridge between Arg113 and Asp93. These residues, which correspond to Arg117 and Thr97 in LamR (FIG. 1a and 1b), project away from the domain in LamR220 and instead pack against a symmetry (two [symbol] fold)-related molecule in the crystal structure. Within this crystallographic dimer, Ala114 packs into a tight pocket in the symmetry-related molecule and Phe116 is in van der Waals contact with Tyr139. We mutated LamR Ala114 to Lys and Phe116 to Ala (abbreviated A114F/K116A), the corresponding residues in S. cerevisiae S2 ribosomal protein ortholog. Wild-type LamR220 bound laminin with an K_{d} of 2.3 μM, and A. fulgidus S2p showed no binding to laminin (Table 1, FIG. 2a). A114F/K116A had no effect on laminin binding (Table 1, FIG. 2a).

TABLE 1
Table 1. Effect of mutations of LamR on laminin binding activity.

| | Half-maximal | Relative |
|---|---|---|
| Mutant | binding [μM] | binding (% age) |
| WT LamR220 | 2.3 +/− 0.16 | 100 |
| L16Q | 2.8 +/− 0.15 | 82 |
| R53A E56A K57A | 1.9 +/− 0.21 | 121 |
| R102A | 2.7 +/− 0.16 | 85 |
| A114K F116A | 3.5 +/− 0.31 | 66 |
| H131A | 5.4 +/− 0.28 | 43 |
| Y139A | 6.2 +/− 0.9 | 37 |
| N141A | 2.2 +/− 0.13 | 105 |
| K166A | 4.3 +/− 0.92 | 53 |
| S190-P194 polyAla | 2.0 +/− 0.17 | 115 |
| F32V | 97.0 +/− 41.8 | 2 |
| E35K | 18.5 +/− 2.9 | 12 |
| R155A | 52.8 +/− 20.8 | 4 |
| F32V E35K | NB | N/A |

Half-maximal binding (K_{d}) was generated using nonlinear regression of 5 binding curves in Prism. Relative binding refers to the ratio of LamR220 wild-type K_{d}/mutant K_{d} expressed as a percentage. NB indicates mutations that result in loss of laminin binding activity.

The second region of structural divergence between human LamR and A. fulgidus S2p is a 5-residue insertion in human LamR, comprising residues Ser190 through Pro194 (FIG. 1b). Mutation of all five residues (Ser190-Pro194) to Ala resulted in no change in laminin binding (Table 1, FIG. 2a) compared to wild type LamR220. In addition, we examined mutation of a previously identified putative laminin binding site, peptide G. Peptide G, residues 161-180, was implicated as a binding epitope in assays that utilized peptide segments of LamR to bind laminin(47). Of this segment, only residues Lys166 and His169 are solvent accessible in the LamR220 crystal structure (46). Mutation of Lys 166 to Ala did not affect laminin binding affinity (Table 1, FIG. 1a), which suggests that Lys166 is not essential for the laminin-binding function of LamR. Lastly, LamR residues Leu16, Phe32, Glu35, Arg53, Glu56, Lys57, Arg102, His131, Tyr139, Asn141, and Arg155 5 was selected (FIGS. 1a and 1c), which are solvent exposed in the LamR220 structure and show sequence conservation among laminin binding species, but are not conserved with the non-binding A. fulgidus S2p. These mutations include changes in charge (for Glu35, Arg53, Glu56, Lys57, Arg102, His131, and Arg155) and in hydrophobicity (for Leu16, Phe32, Tyr139, and N141). Only mutations Phe32, Glu35, and Arg155 resulted in an appreciable loss of binding affinity (Table 1, FIG. 2a). Single point mutation of either LamR Phe32 to Val, the corresponding residue in S. cerevisiae S2 ribosomal protein, Glu35 to Lys or Arg155 to Ala, the corresponding residues in A. fulgidus S2 ribosomal protein, resulted in decreases in laminin binding (8- to 42-fold increase in Kₐ) (Table 1, FIG. 2a). Double point mutation of Phe32 and Glu35 or Phe32 and Arg155 resulted in loss of laminin binding activity (Table 1, FIG. 2a). Mutation of His131, Tyr139, and Asn141, which are in close proximity to Arg155, Phe32, and Glu35, did not affect laminin binding (Table 1, FIG. 1c). These data demonstrate that Phe32, Glu35, and Arg155, located in the face of LamR in the α1-β2 linker region (FIG. 1c), comprise a primary laminin binding site.

To assess the physiological significance of residues Phe32, Glu35, and Arg155 in laminin binding, a cellular assay to test wild-type and mutant LamR function was developed. Purified recombinant LamR was utilized as a soluble decoy to interfere with endogenous LamR cell surface interactions with laminin in a cell migration assay. The ability of recombinant wild-type or mutant LamR220 to inhibit HT-1080 cell migration towards laminin in a Boyden chamber in which cells were added to the top chamber and laminin or laminin with recombinant protein were added to the bottom chamber was analyzed. When recombinant wild-type LamR220 was added with laminin, HT-1080 cell migration towards laminin is 8% of the migration observed relative to migration to laminin alone (FIG. 3a, WT). These data show that recombinant LamR220 interacts with laminin in solution, inhibiting cellular migration towards laminin. Cellular migration to recombinant protein alone is not observed. Protein purity and equal loading was examined by SDS-PAGE. A. fulgidus S2p, which does not exhibit laminin binding activity in vitro, was utilized as a negative control for migration inhibition (FIG. 3a, Rps2). LamR220 mutants F32V/E35K and F32V/R155A, which exhibited loss of laminin binding in vitro, were examined for loss of inhibition and restoration of cell migration to laminin. Both LamR220 mutants F32V/E35K and F32V/R155A demonstrate a significant loss of inhibition of cell migration to laminin (FIG. 3a, F32V/E35K and F32V/R155A). LamR220 F32V/R155A behaves similarly to A. fulgidus S2p (negative control), with 70% migration observed compared to migration to laminin alone. LamR220F32V/E35K results in 46% of migration compared to migration to laminin alone. Cell migration towards 10% FBS was not inhibited by wild-type LamR220, mutant LamR220, or A. fulgidus S2p, which shows that inhibition of cell migration to laminin by wild-type LamR220 is specific to interactions between LamR and laminin (FIG. 3b). Together, these data demonstrate the physiological role of Phe32, Glu35, and Arg155 in laminin binding.

### Example 4

### Synthesis of LRI-F/MRF/Face

### Benzene-1,2,4-triamine (2)

A suspension of 4-nitrobenzene-1,2-diamine 1 (15.3 g, 0.1 mol)¹ in methanol (50 mL) was hydrogenated in a tightly closed vessel in the presence of 10% palladium on carbon (200 mg) under stirring with the use of a magnetic stirrer at a pressure slightly higher than atmospheric until a practically colorless mixture formed and hydrogen absorption ceased. The reaction rate can be controlled by the stirring intensity. ¹ It is possible to use 2,4-dinitroaniline. However, while in the first case the hydrogenation is rather rapid (exothermic), in the second case it requires stirring for several days.

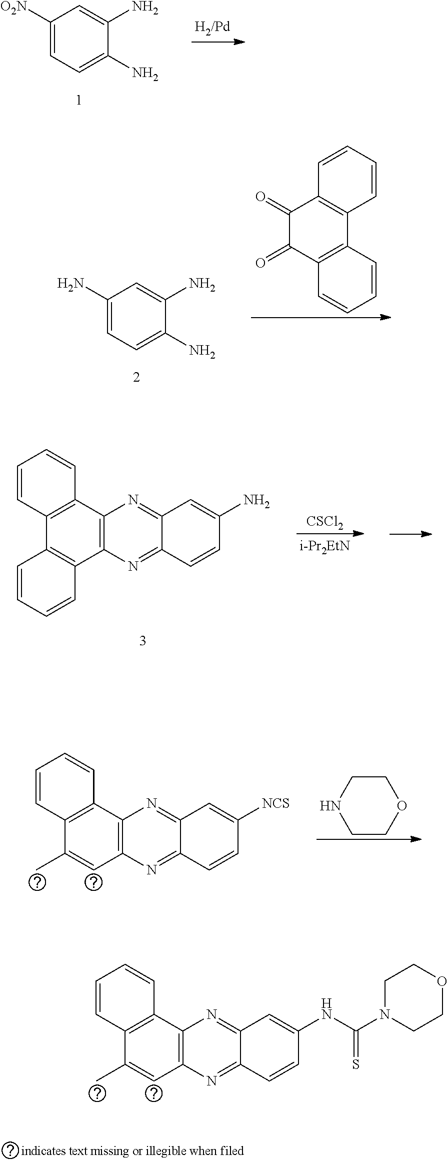

### Dibenzo[a,c]phenazin-11-amine (3)

A solution of phenanthrene-9,10-dione (21.85 g, 1.05 eq) in hot DMSO (150 mL) was added to the obtained mixture under shaking. (Caution! Ebullition of methanol!) The reaction mixture was quickly heated to 150-160° C. to remove methanol and filtered under boiling through a folded paper filter. The hydrogenation vessel and the filter were washed with boiling DMSO (20 mL). The cooled filtrate was diluted with the equal volume of dichloromethane and left for crystallization in a fridge overnight. The precipitated crystals were filtered off and washed with dichloromethane until the filtrate became light. Compound 3 (19.7 g, 67%) was obtained as goldish-brown crystals, practically pure according to TLC. ² Benzene-1,2,4-triamine undergoes instant oxidation in the air. So, it is better to add the solution of phenanthrene-9,10-dione directly into the hydrogenation vessel and quickly heat up the reaction mixture to boiling, which is required to complete the reaction.

### 11-Isothiocyanatodibenzo[a,c]phenazine (4)

Amine 3 (7.76 g, 0.026 mol) was dissolved in boiling dioxane (300 mL). DIPEA (7.71 g, 2.3 eq) was added to the solution. A solution of thiophosgene (3.3 g, 1.1 eq) in dioxane (20 mL) was added dropwise under stirring with the use of a magnetic stirrer at 60-70° C. for 10-15 min. The obtained solution was diluted with dichloromethane (50 mL). After recrystallization overnight in a fridge and washing with ice-cold dichloromethane (30 mL) a product (6.42 g) pure according to TLC was obtained. Evaporation of the filtrate to 40 mL, addition of dichloromethane (40 mL) to the residue, and repeated crystallization gave 1.56 g more of the pure substance. Overall yield of compound 4: 7.98 g (>90%).

### N-Dibenzo[a,c]phenazin-11-ylmorpholine-4-carbothioamide (5)

Isothiocyanate 4 was dissolved in hot dioxane, and morpholine (1.05 eq) was added³. Filtration of the reaction mixture gave the pure thiourea in practically quantitative yield. Trace amounts of dioxane can be removed from the product by boiling in ethanol. ³ The addition time is determined only by the amount of the reagents. The reaction proceeds instantly, but requires good mechanical stirring (the reaction mixture is very thick).

### Example 5

### Virtual Ligand Screening

The crystal structure of human LamR (resolved residues 9-205, PDB Code: 3BCH) was accessed using MolSoft ICM software (MolSoft) and converted to an ICM Object for generating a virtual model in preparation for screening. Population of hydrogens, removal of all water molecules, assignment of amino acid charges and optimization of hydrogen, histidine, asparagine, glutamine and proline residue positions and isomeric states was performed. With the structure prepared in this manner, virtual ligand screening (VLS) was performed by the internal coordinate mechanics (ICM) method. Receptor maps were made to include residues L25-G27, D126-R128, N149-S152 and N164-K166 for the docking method (ECEPP/3 and MMFF based system using a biased probability Monte Carlo method for pose searching). A virtual library of approximately 500,000 small molecules (ChemBridge) was screened in triplicate using this process with the same setup parameters conserved for each replicate. The best ICM docking scores (ICMScore) from each independent screen were used to evaluate probability of binding and aid in selection of hits.

### PAPER EXAMPLE 1

### Testing the Compounds of the Invention in a Mouse Model of Alzheimer's Disease

Testing of the compounds of the present invention for their effectiveness in treating Alzheimer's Disease will be performed using the Tg2576 APP mouse model developed by Karen Hsiao and colleagues (77). These mice develop Abeta plaques as early as at 11 to 13 months of age. The mice will be tested in the well-known Radial Arm Maze as follows.

The mice will receive treatment with 0.42 mg/20 g of Face injected subcutaneously. The mice will be maintained on a 12-hour light-dark cycle, and have access to food and water ad libitum. The animal care will be in accordance with institutional guidelines. Animals will be kept in a test room throughout the experiment, behind a cover to prevent view of the apparatus and room. Each animal will undergo 2 days of adaptation, consisting of 15 minutes of maze exploration (2 subjects at a time), with 3 pieces of fruit loops in each arm. Subjects will be exposed to arm doors only on day 2. Animals will be food-deprived 1 day before the first adaptation session and maintained at approximately ten percent body weight loss. Fruit loops will be added to normal diet 5 days before deprivation schedule starts. Animals will enter and exit the apparatus through the center of the maze. Testing will include recording correct and incorrect arms entered. Each trial will be initiated by placing the mouse in the center of the maze and all doors into the arms will be subsequently opened. After entry into an arm, the animal will have to find and eat the reinforcer before the door will be reopened to allow the animal to re-enter the center of the maze. Testing will end when all eight arms are entered and reinforcers eaten. Re-entry into an arm constitutes an error. Total number of errors and time to enter all eight arms will be recorded. The animals will be allowed access to food for up to 3-4 hours daily, depending on their body weight loss. The corners and holes in the maze will be cleaned with 95% ethanol after each animal enters and leaves the arms.

It is expected that the mice which receive the compounds described herein will finish the maze sooner and with fewer errors than untreated controls.

### REFERENCES

1. Rao, N. c., Barsky, S. H., Terranova, V. P., and Liotta, L. A. (1983) Biochem Biophys Res Commun 111, 804-808
2. Terranova, V. P., Rao, C. N., Kalebic, T., Margulies, l. M., and Liotta, L. A. (1983) Proc Natl A cad Sci USA 80, 444-448
3. Malinoff, H. L., and Wicha, M. S. (1983) J Cell Bioi 96, 1475-1479
4. Mecham, R. P. (1991) Faseb J 5, 2538-2546
5. Smyth, N., Vatansever, H. S., Murray, P., Meyer, M., Frie, c., Paulsson, M., and Edgar, D. (1999) J Cell Bioi 144, 151-160
6. Grat: l., Ogle, R c., Robey, F. A., Sasaki, M., Martin, G. R., Yamada, Y., and Kleinman, H. K. (1987) Biochemistry 26, 6896-6900
7. Narumi, K., Inoue, A., Tanaka, M., lsemura, M., Shimo-Oka, T., Abe, T., Nukiwa, T., and Satoh, K. (1999) Jpn J Cancer Res 90, 425-431
8. Satoh, K., Narumi, K., Isemura, M., Sakai, T., Abe, T., Matsushima, K., Okuda, K., and Motomiya, M. (1992) Biochem Biophys Res Commun 182, 746-752
9. Martignone, S., Menard, S., Bufalino, R, Cascinelli, N., Pellegrini, R, Tagliabue, E., Andreola, S., Rilke, F., and Colnaghi, M. I. (1993) J Natl Cancer Inst 85, 398-402
10. de Manzoni, G., Guglielmi, A., Verlato, G., Tomezzoli, A., Pelosi, G., Schiavon, I., and Cordiano, C. (1998) Oncology 55, 456-460
11. Cioce, V., Castronovo, V., Shmookler, B. M., Garbisa, S., Grigioni, W. F., Liotta, L. A., and Sobel, M. E. (1991) J Natl Cancer Inst 83, 29-36
12. Sanjuan, X., Fernandez, P. L., Miguel, R, Munoz, J., Castronovo, V., Menard, S., Palacin, A., Cardesa, A., and Campo, E. (1996) J Path 01179, 376-380
13. Liebman, l. M., Burbelo, P. D., Yamada, Y., Fridman, R., and Kleinman, H. K. (1993) Int J Cancer 55, 102-109
14. van den Brule, F. A., Castronovo, V., Menard, S., Giavazzi, R, Marzola, M., Belotti, D., and Taraboletti, G. (1996) Eur J Cancer 32A, 1598-1602
15. van den Brule, F. A., Buicu, C., Berchuck, A., Bast, R C., Deprez, M., Liu, F. T., Cooper, D. N., Pieters, C., Sobel, M. E., and Castronovo, V. (1996) Hum Pathol 27. 1185-1191
16. Montuori, N., Muller, F., De Riu, S., Fenzi, G., Sobel, M. E., Rossi, G., and Vitale, M. (1999) J Clin Endocrinol Metab 84, 2086-2092
17. Waltregny, D., de Leval, L., Menard, S., de Leval, l., and Castronovo, V. (1997) J Natl Cancer Ins! 89, 1224-1227
18. Ozaki, l., Yamamoto, K., Mizuta, T., Kajihara, S., Fukushima, N., Setoguchi, Y., Morito, F., and Sakai, T. (1998) Gut 43, 837-842 11
19. Vacca, A, Ribatti, D., Roncali, l., Lospalluti, M., Serio, G., Carrel, S., and Dammacco, F. (1993) Cancer 72, 455-461
20. Taraboletti, G., Belotti, D., Giavazzi, R., Sobel, M. E., and Castronovo, V. (1993) J Nat! Cancer Inst 85, 235-240
21. Menard, S., Tagliabue, E., and Colnaghi, M. I. (1998) Breast Cancer Res Treat 52, 137-145
22. Castronovo, V. (1993) Invasion Metastasis 13, 1-30
23. Liotta, L. A., Rao, N. c., Barsky, S. H., and Bryant, G. (1984) Ciba Found Symp 108, 146162
24. Aznavoorian, S., Murphy, A N., Stetler-Stevenson, W. G., and Liotta, L. A (1993) Cancer 71, 1368-1383
25. Liotta, L. A (1986) Cancer Research 46, 1-7
26. Lee, W. A., Kim, W. H., Kim, Y. I., Yang, H. K., Kim, I P., and Kleinman, H. K. (1996) Pathol Res Pract 192, 1195-1201
27. Castronovo, V., Taraboletti, G., Liotta, L. A, and Sobel, M. E. (1989) J Natl Cancer Inst 81, 781-788 10
28. Wang, K. S., Kuhn, R. l, Strauss, E. G., Qu, S., and Strauss, l H. (1992) J Virol 66, 49925001
29. Ludwig, G. V., Kondig, J. P., and Smith, J. F. (1996) J Viro/70, 5592-5599
30. Akache, B., Grimm, D., Pandey, K., Yant, S. R., Xu, H., and Kay, M. A. (2006) J Viro/80, 9831-9836
31. Tachibana, H., Koga, K., Fujimura, Y., and Yamada, K. (2004) Nat Struct Mol Bioi 11, 380381
32. Ardini, E., Pesole, G., Tagliabue, E., Magnifico, A, Castronovo, V., Sobel, M. E., Colnaghi, M. I., and Menard, S. (1998) Mol Bioi Evo/15, 1017-1025
33. Auth, D., and Brawerman, G. (1992) Proc Nall Acad Sci USA 89, 4368-4372
34. Scheiman, J., Tseng, J. C., Zheng, Y., and Meruelo, D. (2010) Mol Ther 18, 63-74
35. Sato, M., Kinoshita, K., Kaneda, Y., Saeki, Y., Iwamatsu, A, and Tanaka, K. (1996) Biochern Biophys Res Cornmun 229, 896-901
36. Davis, S. C., Tzagoloff, A, and Ellis, S. R. (1992) J Bioi Chem 267, 5508-5514
37. Garcia-Hernandez, M., Davies, E., and Staswick, P. E. (1994) J Bioi Chem 269, 20744-20749
38. Keppel, E., and Schaller, H. C. (1991) J Cell Sci 100 (Pt 4), 789-797
39. Rosenthal, E. T., and Wordeman, L. (1995) J Cell Sci 108 (Pt 1), 245-256
40. Melnick, M. B., Noll, E., and Perrimon, N. (1993) Genetics 135, 553-564
41. Lopez-Ribot, J. l., Casanova, M., Monteagudo, C., Sepulveda, P., and Martinez, J. P. (1994) Infect Irnrnun 62, 742-746
42. Zhang, L., Leggatt, G. R., Kalinna, B. H., Piva, T. J., and McManus, D. P. (1997) Mol Biochern Parasitol 87, 183-192
43. Buto, S., Tagliabue, E., Ardini, E., Magnifico, A, Ghirelli, c., van den Brule, F., Castronovo, V., Colnaghi, M. I., Sobel, M. E., and Menard, S. (1998) J Cell Biochem 69, 244-251
44. Gauczynski, S., Peyrin, J. M., Haik, S., Leucht, C., Hundt, C., Rieger, R., Krasemann, S., Deslys, J. P., Dormont, D., Lasmezas, C. I., and Weiss, S. (2001) Embo J 20, 5863-5875
45. Fatehullah, A, Doherty, C., Pivato, G., Allen, G., Devine, L., Nelson, J., and Timson, D. J. (2010) Biosci Rep 30, 73-79
46. Jamieson, K. V., Wu, J., Hubbard, S. R., and Meruelo, D. (2008) J Bioi Chem 283, 30023005
47. Castronovo, V., Taraboletti, G., and Sobel, M. E. (1991) J Bioi Chem 266, 20440-20446 12
48. Landowski, T. H., Uthayakumar, S., and Starkey, l. R. (1995) Clin Exp Metastasis 13, 357372
49. Kazmin, D. A., Hoyt, T. R., Taubner, L., Teintze, M., and Starkey, l. R. (2000) J Mol Bioi 298, 431-445
50. Badger, l., Sauder, l. M., Adams, l. M., Antonysamy, S., Bain, K., Bergseid, M. G., Buchanan, S. G., Buchanan, M. D., Batiyenko, y, Christopher, J. A., Emtage, S., Eroshkina, A., FeU, I., Furlong, E. B., Gajiwala, K. S., Gao, x., He, D., Hendle, l., Huber, A., Hoda, K., Kearins, P., Kissinger, c., Laubert, B., Lewis, H. A., Lin, l., Loomis, K., Lorimer, D., Louie, G., Maletic, M., Marsh, C. D., Miller, l., Molinari, l., MullerDieckmann, H. l., Newman, J. M., Noland, B. W., Pagarigan, B., Park, F., Peat, T. S., Post, K. W., Radojicic, S., Ramos, A., Romero, R., Rutter, M. E., Sanderson, W. E., Schwinn, K. D., Tresser, l., Winhoven, l., Wright, T. A., Wu, L., Xu, l., and Harris, T. l. (2005) Proteins 60, 787-796
51. Chow, H. H., Cai, y, Alberts, D. S., Hakim, I., Dorr, R., Shahi, F., Crowell, L A., Yang, C. S., and Hara, Y. (2001) Cancer Epidemiol Biomarkers Prey 10, 53-58
52. Wang, Y. C., and Bachrach, U. (2002) Amino Acids 22, 131-143
53. Lung, H. L., Ip, W. K., Wong, C. K., Mak, N. K., Chen, Z. y, and Leung, K. N. (2002) Life Sci 72, 257-268
54. Lung, Y. D., and Ellis, L. M. (2001) Int l Exp Path 01 82, 309-316 11
55. Masuda, M., Suzui, M., Lim, l. T., and Weinstein, l. B. (2003) Clin Cancer Res 9, 3486-3491
56. Shammas, M. A., Neri, P., Koley, H., Batchu, R. B″ Bertheau, R. C., Munshi, V., Prabhala, R., Fulciniti, M., Tai, Y. T., Treon, S. P., Goyal, R. K., Anderson, K. C., and Munshi, N. C. (2006) Blood 108, 2804-2810
57. Fassina, G., Vene, R., Morini, M., Minghelli, S., Benelli, R., Noonan, D. M., and Albini, A. (2004) Clin Cancer Res 10, 4865-4873
58. Lung, Y. D., Kim, M. S., Shin, B. A., Chay, K. 0., Ahn, B. W., Liu, W., Bucana, C. D., Gallick, G. E., and Ellis, L. M. (2001) Br J Cancer 84, 844-850
59. Nelson, l., McFerran, N. V., Pivato, G., Chambers, E., Doherty, C., Steele, D., and Timson, D. l. (2008) Biosci Rep 28, 33-48
60. Reich, R., Vintman, L., Nielsen, S., Kaern, l., Bedrossian, C., Berner, A., and Davidson, B. (2005) Diagn Cytopathol 33, 332-337
61. Zuber, C., Knackmuss, S., Zemora, G., Reusch, V″ Vlasova, E., Diehl, D., Mick, V., Hoffmann, K., Nikles, D., Frohlich, T., Arnold, G. l., Brenig, B., Wolf, E., Lahm, H., Little, M., and Weiss, S. (2008) J Mol Bioi 378, 530-539
62. Montuori, N., and Sobel, M. E. (1996) Curr Top Microbiollmmunol 213 (Pt 1), 205-214
63. Magnifico, A., Tagliabue, E., Buto, S., Ardini, E., Castronovo, V″ Colnagbi, M. l., and Menard, S. (1996) J Bioi Chem 271, 31179-31184
64. Pupa, S. M., Menard, S., Forti, S., and Tagliabue, E. (2002) J Cell Physio/192, 259-267
65. Graf, l., Iwamoto, Y., Sasaki, M., Martin, G. R., Kleinman, H. K., Robey, F. A., and Yamada, Y. (1987) Cell 48, 989-996
66. Berno, V., Porrini, D., Castiglioni, F., Campiglio, M., Casalini, P., Pupa, S. M., Balsari, A., Menard, S., and Tagliabue, E. (2005) Endocr Relat Cancer 12, 393-406
67. Tanaka, M., Narumi, K., Isemura, M., Abe, M., Sato, Y., Abe, T., Saijo, Y., Nukiwa, T., and Satoh, K. (2000) Cancer Lett 153, 161-168
68. Abagyan, R. A., Totrov, M. M., Kuznetsov, D. A. 1994 J. Comp. Chem. 15:488-506.
69. Abagyan, R. A., Totrov, M. M, 1994 J. Mol. Biol. 235:983-1002.
70. Totrov, M. M., Abagyan, R. A. 1994 Nature Struc. Biol. 1:259-263.
71. Totrov, M., and Abagyan, 1997 Proteins 1 (Suppl.), 215-220.
72. Bronfman, F. C., J. Gamido, A. Alvarez, C. Morgan & N. C. Inestrosa, (1996). Neuroscience letters 218: 201-203.
73. Castillo, G. M., W. Lukito, E. Peskind, M. Raskind, D. A. Kirschner, A. G. Yee & A. D. Snow, (2000). J Neurosci Res 62: 451-462.
74. Drouet, B., M. Pinson-Raymond, J. Chambaz & T. Pillot, (1999. J Neurochem 73: 742-749.
75. Rieger, R., F. Edenhofer, C. I. Lasmezas & S. Weiss, (1997) Nat Med 3: 1383-1388.
76. Shin, S. J., S. E. Lee, J. H. Boo, M. Kim, Y. D. Yoon, S. I. Kim & I. Mook-Jung, (2004). Proteomics 4: 3359-3368
77 Hsiao, K., Chapman, P., Nilsen, S., Eckman, C., Harigaya, Y., Younkin, S., Yang, F., Cole, G. (1996). Science. 274(5284): 99-102.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed is:

1. A pharmaceutical formulation for treating a mammal suffering from a disease mediated by the laminin receptor (LamR) comprising an agent selected from N-dibenzo[a,c]phenazin-11-yl-4-morpholinecarbothioamide), and N-(2-hydroxyethyl)dibenzo[a,c]phenazine-11-carboxamide. and a pharmaceutically acceptable carrier or diluent.

2. The pharmaceutical formulation of claim 1 wherein said disease is selected from a tumor, Alzheimer's disease, and prion disease.

3. A method for treating a mammal suffering from a tumor comprising administering to a mammal in need of such treatment an amount of an agent selected from N-(2-hydroxyethyl)dibenzo[a,c]phenazine-11-carboxamide and N-dibenzo[a,c]phenazin-11-yl-4-morpholinecarbothioamide). effective to treat said tumor and a pharmaceutically acceptable carrier or diluent.

4. The method of claim 3 wherein said agent is administered systemically.

5. The method of claim 4 wherein said agent is administered parenterally.

6. The method of claim 3 wherein said mammal is a human.

7. The method of claim 3 wherein said tumor expresses greater levels of LamR than normal cells of the same lineage.

8. A method for reducing metastatic growth of a tumor in a mammal comprising administering to a mammal harboring a tumor and in need of such treatment an amount of an agent selected from N-(2-hydroxyethyl)dibenzo[a,c]phenazine-11-carboxamide and N-dibenzo[a,c]phenazin-11-yl-4-morpholinecarbothioamide). effective to reduce metastatic growth of said tumor in said mammal and a pharmaceutically acceptable carrier or diluent.

9. The method of claim 8 wherein said agent is administered systemically.

10. The method of claim 9 wherein said compound is administered parenterally.

11. The method of claim 8 wherein said mammal is a human.

12. The method of claim 8 wherein said tumor expresses greater levels of LamR than normal cells of the same lineage.

13. A method for treating a patient suffering from Alzheimer's disease comprising administering to a patient in need of such treatment an amount of an agent effective to treat Alzheimer's disease selected from N-(2-hydroxyethyl)dibenzo[a,c]phenazine-11-carboxamide and N-dibenzo[a,c]phenazin-11-yl-4-morpholinecarbothioamide and a pharmaceutically acceptable carrier or diluent.

14. The method of claim 13 wherein said agent is administered systemically.

15. The method of claim 14 wherein said agent is administered parenterally.
